(19) United States
(12) Patent Application Publication
(10) Pub. No.: US 2015/0087579 A1
(43) Pub. Date: Mar. 26, 2015

(54) ANTIMICROBIAL PEPTIDES

(71) Applicant: ROBERT BOSCH GENSELLSCHAFT FÜR MEDIZINISCHE FORSCHUNG MBH, Stuttgart (DE)

(72) Inventors: Eduard Stange, Stuttgart (DE); Bjoern Schroeder, Stuttgart (DE); Jan Wehkamp, Stuttgart (DE)

(73) Assignee: ROBERT BOSCH GENSELLSCHAFT FÜR MEDIZINISCHE FORSCHUNG MBH, Stuttgart (DE)

(21) Appl. No.: 14/383,549

(22) PCT Filed: Mar. 7, 2013
(86) PCT No.: PCT/EP2013/054599

§ 371 Date: Sep. 6, 2014

(30) Foreign Application Priority Data
Mar. 7, 2012 (DE) 102012203547.8

(51) Int. Cl. C07K 7/06

(52) U.S. Cl. CPC C07K 7/06; A61K 38/00
USPC 514/2.4; 530/328; 536/23.5

(57) ABSTRACT

A novel antimicrobial peptide includes at least eight successive amino acids, the peptide exhibiting a sequence having the following formula: Ter₁-X₁-B₁-X₂-B₂-X₃-Z₁-Z₂-X₄-Ter₂. The peptide can moreover also have modified termini. The peptide is believed to be effective for the treatment or prevention of inflammatory and infectious diseases that are caused by microorganisms such as bacteria or fungi.

### FIELD OF THE INVENTION

The present invention relates to novel antimicrobial peptides and to the utilization thereof in medicine.

### BACKGROUND INFORMATION

Antimicrobial peptides, also referred to simply as “AMPs,” are part of the natural immune system and are vitally important for epithelial defense against infection by microorganisms.

In a healthy person the skin and mucosa form a physical barrier to infection by microorganisms. The physical barrier is made up of the stratum corneum in healthy skin and, in the mucosa, of the mucous layer in which desquamation and mucous secretion cause a constant renewal of the surfaces, simultaneously with continuous elimination of microorganisms that are adhering to the surfaces. In interaction with the lipids that are also present in the skin, this physical barrier prevents microorganisms from penetrating into the living epidermis.

Leaving aside this physical barrier, however, further factors are also necessary in order for the healthy skin and mucosa to defend against infection; among these factors are endogenous antimicrobial peptides. Lysozyme, for example, is an antimicrobial peptide that is present in nasal secretions and can in particular kill Gram-positive bacteria. Also known as antimicrobial peptides in the intestinal mucosa are defensins, whose presence appears to be necessary especially given that the intestinal epithelia are exposed to very large quantities of bacteria. In addition to having a mucous layer that is difficult for microorganisms to penetrate, the intestinal mucosa contains paneth cells that secrete human defensin-5 and, among other functions, protect the stems cells that are important for continuous renewal of the intestinal mucosa.

Further known AMPs are a peptide known as psoriasin, as well as RNas-7, which represents an effective endogenous broad-spectrum antibiotic in humans.

In addition to the known endogenous antimicrobial peptides, numerous antibiotics are also known in the existing art; these include both substances of biological origin and synthetically manufactured substances, which are therefore either (as in the original sense) naturally formed low-molecular-weight metabolic products of fungi or bacteria, or chemically synthesized therapeutic agents.

Especially in light of the fact that the development of resistance to natural and synthetic antibiotics is making microbial infectious diseases increasingly difficult to treat, a need also frequently arises for novel antimicrobial active agents that are notable for few side effects and for simple manufacture and handling.

### SUMMARY OF THE INVENTION

In light of this, an object of the present invention is to furnish a novel antimicrobial substance that can be used to treat infectious microbial diseases.

This object is achieved according to the present invention by a peptide that has antimicrobial activity and has a C-terminus and an N-terminus, and that is made up of at least eight and at most 12 successive amino acids, the peptide exhibiting the sequence having the following formula I:

Ter₁-X₁-B₁-X₂-B₂-X₃-Z₁-Z₂-X₄-Ter₂ (formula I)
in which
Ter₁ is the free N-terminal amino group of the N-terminal amino acid X₁, or a modified N-terminal amino group;
X₁, X₂, and X₃ are each identical or different and are selected, mutually independently in each case, from an amino acid having a basic side chain, which may be are selected from one of the following: arginine, lysine, 6-hydroxylysine, homoarginine, 2,4-diaminobutyric acid, [beta]-homoarginine, D-arginine, arginal, 2-amino-3-guanidinopropionic acid, nitroarginine, n-methylarginine, [epsilon]-n-methyllysine, allo-hydroxylysine, 2,3-diaminopropionic acid, 2,2′-diaminopimelic acid, ornithine, sym-dimethylarginine, asym-dimethylarginine;
B₁ and B₂ are identical or different and are selected, mutually independently in each case, from an amino acid having an aliphatic or basic side chain, and may be selected from alanine or glycine;
Z₁ and Z₂ either are each cysteine, or are cysteine and alanine; and
Ter₂ is the free C-terminal carboxyl group of the C-terminal amino acid X₄, or is a modified C-terminal carboxyl group.

It may be provided here that the peptide is made up of eight amino acids, and possesses a sequence of formula I.

As already stated, Z₁ and Z₂ either are each cysteine, or are cysteine and alanine; i.e. if Z₁ is cysteine then Z₂ is alanine, and if Z₁ is alanine Z₂ is cysteine.

The peptide may be manufactured synthetically, manufactured recombinantly, obtained by enzymatic cleavage, and/or isolated. Since the peptide according to the present invention is a relatively short peptide, it may be the case that the peptide according to the present invention is manufactured synthetically; synthetic manufacturing methods are sufficiently known in the existing art and encompass in particular liquid-phase and solid-phase chemical synthesis methods. Reference is made by way of example to the review article and standard work S. Kent, “Chemical Synthesis of Peptides and Proteins,” Annual Review of Biochemistry 57:957-989 (1988). Numerous companies that commercially manufacture synthetic peptides are also active at present in the relevant sector.

Besides the eight amino acids of formula I, the peptide according to the present invention can have at both the N-terminus and the C-terminus further amino acids that do not, or that only slightly, impair the effectiveness and stability of the peptide according to the present invention. It will be clear to one skilled in the art, proceeding from the structure of the present peptide according to the present invention, which amino acids or amino acid residues can additionally be attached at the C- or N-terminus in order to allow achievement of an antimicrobial effect identical or very similar to that of the peptide made up of eight amino acids.

In the inventors' own experiments, the peptide according to the present invention proved to be extremely effective with respect to a number of bacterial and fungal strains.

The term “peptide” is understood here as a sequence of amino acids that are each linked to one another via peptide bonds; the amino acids may be selected from the twenty naturally occurring amino acids, and the amino acids can be present therein in the L-configuration or D-configuration. Alternatively to the peptide and proceeding from its mode of operation and structure, it is also possible to manufacture peptidomimetics that according to the present invention are therefore also encompassed by the present invention.

Peptidomimetics are in this present case, by definition, low-molecular-weight chemical compounds whose essential structural elements are modeled on the peptide according to the present invention. The peptide according to the present invention can be present, for example, in isolated, synthetic, or recombinant form, or can be made available in corresponding form.

The term “antimicrobial” is understood in the present case as the property of being able to reduce the reproductive ability or infectiousness of microorganisms, or to kill or inactivate them. “Microorganisms” are understood as microscopically small organisms or units that usually are not detectable with the naked eye, and in the present case are understood in particular as bacteria, viruses, and fungi that cause processes deleterious to health (diseases) in other organisms, in particular in humans or other mammals.

According to an exemplary embodiment, the peptide according to the present invention is selected from one of SEQ ID nos. 1 to 6 or derivatives thereof, the derivatives being formed by exchanging at least one amino acid with a derivative of the amino acid. The following peptides may be used in particular: the peptide having the sequence RGKAKCCK (SEQ ID no. 1), the peptide having the sequence RGKAKCAK (SEQ ID no. 2), and the peptide having the sequence RGKAKACK (SEQ ID no. 3), specifically in unmodified form, i.e. with unmodified termini, or in modified form, i.e. having at least one modified (C- or N-) terminus, or in modified form having a modified N-terminus and a modified C-terminus (see SEQ ID nos. 4, 5, and 6).

The term “derivative of the/an amino acid” is to be understood to mean all amino residues derived from the respective amino acid that are obtained from the respective amino acid e.g. by structural modification of a functional group.

The term “modified N-terminal amino group” and “modified C-terminal carboxyl group” are understood here as a modified amino group or carboxy group. Examples of N-terminal modifications are acetylated, formylated, or guanylated N-termini. Examples of C-terminal modifications are amidated C-termini.

It particularly may be that the peptide is made up in each case entirely of D-amino acids or L-amino acids or of mixtures thereof. In the present case, “D-amino acids” or “L-amino acids” means that the natural amino acids, unnatural amino acids, or amino acid derivatives (such as imino acids) to be used can be present in the L- or the D-configuration.

According to a further embodiment it may be the case that the peptide is modified at the C-terminus and/or at the N-terminus, and in particular is modified by an acetylation, amidation, formylation, or guanylation.

The modification of the C- and/or N-termini of the peptides according to the present invention has the advantage that as a result they are more stable with regard to breakdown by peptidases and proteases; the peptides according to the present invention thus have an extended half-life time in, for example, serum. The modifications of the N- and C-termini also permit coupling of the peptides to other groups, for example to other amino acid sequences or other biomolecules.

In a further embodiment of the peptide according to the present invention, it is reduced or is present in an oxidized state.

According to the present invention the peptide is used for the treatment and/or prophylaxis of inflammatory or infectious diseases that are caused by microorganisms.

According to the present invention the use therefore occurs in the context of inflammatory and/or infectious diseases that are caused by bacteria, viruses, or fungi.

The use according to the present invention occurs in particular in the context of inflammatory or infectious diseases that are caused by a microorganism that is selected from Bifidobacterium sp., Lactobacillus sp., Escherichia coli, Streptococcus sp., Staphylococcus sp., Bacteroides sp., Candida sp., Pseudomonas sp., Propionibacterium sp., Treponema sp., Enterobacter sp., Salmonella sp., Legionella sp., it being understood that this list is not exhaustive and that the peptide is also effective against bacterial and/or fungal strains not set forth herein and in particular against bacteria that belong in general to the family of Neisseriaceae, Enterobacteriaceae.

It particularly may be that the use of the peptide according to the present invention occurs in the context of chronic inflammatory intestinal diseases, inflammatory diseases of the oropharyngeal cavity, for example caries and gingival inflammations, pulmonary diseases, diseases of the urogenital tract, diseases of the pancreas, diseases of the female reproductive system, diseases of and/or injuries to the skin (dermatological diseases).

The present invention correspondingly also relates to a pharmaceutical composition that has at least one peptide according to the present invention as well as optionally a pharmaceutically acceptable carrier and further formulation substances and adjuvants usual in the existing art, and to a method for treating mammals that are suffering from inflammatory infectious diseases caused by microorganisms, in which method a therapeutically effective quantity of the peptide according to the present invention or of the pharmaceutical composition according to the present invention is administered. “Therapeutically effective” or a “therapeutically effective quantity” means here that quantity of the at least one peptide according to the present invention, or of the pharmaceutical composition that has at least one peptide according to the present invention, which is capable of reducing or entirely preventing reproduction and colony formation of the bacteria and/or fungi, or of achieving a measurable therapeutic or prophylactic success. The exact effective quantity for a subject depends on its size and state of health, on the nature and extent of the disease, and on the at least one peptide or pharmaceutical composition or combination of several aforesaid thereof.

The formulations/medications of the present invention can be utilized either in vitro or in vivo.

The pharmaceutical compositions of the present invention can be administered to a patient in a plurality of forms that are adapted to selected route of administration, namely parenteral, oral, intraperitoneal, transdermal, etc. Parenteral administration here includes administration by the following routes: intravenous, intramuscular, interstitial, intraarterial, subcutaneous, intrasynovial, transepithelial including transdermal, pulmonary via inhalation, ophthalmic, sublingual and buccal, topical including ophthalmic, dermal, ocular, rectal, and nasal inhalation via insufflation.

Administration can occur in the form of solutions, tinctures, salves, powders, suspensions, creams, and further solid or liquid formulations, and as tablets, capsules, spray.

Included among the diseases of the skin that can be treated with the present peptide according to the present invention or with a medication containing it are, for example, acne, dermatitis, burns, and other skin diseases that have been caused by microorganisms, or in the context of injuries to the skin in which the risk of a microbial infection exists.

According to an exemplary embodiment the pharmaceutical composition is administered through or via the skin, which represents a noninvasive and patient-friendly administration and has the advantage, as compared with oral administration, that the medium in the digestive system need not be considered. Uptake through the skin is possible, for example, in the nose, the cheek, under the tongue, on the gums, or in the vagina. Corresponding presentation forms can be achieved using known techniques; they can be processed into nose drops, nasal spray, inserts, films, patches, gels, suppositories, salves, or tablets. The excipient for uptake through the skin may contain one or more components that adhere to the skin and thereby extend the contact time between the presentation form and the adsorbing surface, in order thereby to increase uptake by absorption. The at least one peptide according to the present invention can thus be formulated, for example, in liposomes that assist introduction of the peptide into the skin.

The peptide according to the present invention can furthermore be used to treat diseases of the oropharyngeal cavity, and in such uses can be present in the form of toothpastes, mouthwashes, gels, and/or e.g. on dental floss.

As already mentioned previously, the pharmaceutical composition can also contain, besides the at least one peptide according to the present invention, two or more of the peptides according to the present invention. The pharmaceutical composition can moreover also contain, besides the at least one peptide according to the present invention, one or more further active substances, for example antibiotics known in the existing art (e.g. streptomycin, penicillin, tetracycline) or other antimicrobially active compounds such as fungicides, for example miconazole, or other substances with which the symptoms associated with an infection, e.g. fever or skin rash, are usually treated.

The medication can in addition also contain pharmaceutically acceptable carriers, binding agents, excipients, or adjuvants. A pharmaceutical carrier, excipient, or diluent can be selected with regard to the intended route of administration and standardized pharmaceutical practice. Pharmaceutically acceptable carriers that can be used are solvents, extending agents, or other liquid binding agents such as dispersion or suspension adjuvants, surface-active agents, isotonic active agents, thickening agents or emulsifiers, preservatives, encapsulating agents, solid binding materials, or slip agents, depending on what is most suitable for the particular dosage and at the same time is compatible with the peptide. The pharmaceutical composition can also contain buffers, diluents, and/or additives. Suitable buffers include, for example, Tris-HCl, glycine, and phosphate, and suitable diluents include e.g. aqueous NaCl solutions, lactose, or mannitol. Suitable additives include, for example, detergents, solvents, antioxidants, and preservatives and protective colloids, for example homologous albumen or biocompatible hydrogels. An overview of such additional ingredients may be found, for example, in A. Kibbe: “Handbook of Pharmaceutical Excipients,” 3rd ed., 2000, American Pharmaceutical Association and Pharmaceutical Press.

Furthermore, the pharmaceutical composition according to the present invention can also have pharmaceutically acceptable salts, for example salts of mineral acids such as hydrochlorides, hydrobromides, phosphates, sulfates, and comparable ones; but also salts of organic acids, such as acetates, propionates, malonates, benzoates, and comparable ones.

In general, a therapeutically effective daily dose will presumably be in the range from 0.01 to 50 mg per kg of body weight of the subject to be treated, which may be from 0.1 to 20 mg/kg. As also previously mentioned above, the medication can be furnished in the form of tablets or capsules, which can be administered singly or two or more thereof simultaneously. The medication can also be furnished in the form of a delayed-release formulation.

The physician will typically determine the daily dose suitable for a specific patient, which will depend on his or her age, weight, and the patient's general state of health.

Depending on utilization, the medication can be administered by inhalation, in the form of a suppository or pessary, topically as a solution, lotion, salve, cream, or loose powder, with the use of a skin patch, orally in the form of tablets or capsules, elixirs, solutions, or suspensions, which optionally can contain flavors or coloring agents.

In addition to therapeutic use for the treatment of infections, the at least one peptide according to the present invention can also be use in disinfecting agents or cleaning agents that can be utilized for disinfection or cleaning of surfaces or objects. Another area of utilization is packages, in which peptides can be bound to the packaging material or incorporated thereinto, or as preservatives for other materials that can easily be broken down by microorganisms.

In addition to utilization of the peptide according to the present invention in human medicine, utilization in veterinary medicine is also possible.

The present invention further relates to an isolated nucleic acid molecules whose sequence codes for the peptide according to the present invention and in particular for a peptide having SEQ ID nos. 1 to 6 that denotes the antimicrobial, i.e. antibacterial or antimycotic, peptide or coding nucleic acid according to the present invention, in operative connection with a regulatory sequence that controls its expression in the host cell. A further constituent of the invention is a host cell that is transfected or transformed with the above-described nucleic acid molecule.

Further advantages are evident from the description below and from the attached Figures.

It is understood that the features recited above and those yet to be explained below are usable not only in the respective combination indicated, but also in other combinations or in isolation, without departing from the scope of the present invention.

Exemplifying embodiments of the invention are depicted in the drawings and will be explained in further detail in the description that follows.

### BRIEF DESCRIPTION OF THE DRAWINGS

FIG. 1 shows the results of investigations of the antimicrobial effect of various peptides (heptapeptides (a); octapeptides (b) (c)) with respect to the bacteria Bifidobacterium adolescentis or Escherichia coli. The letters indicate the amino acids using the single-letter code. The peptide ac-RGKAKCCK-NH₂ (c) (SEQ ID no. 4) possesses an acetylated amino terminus and an amidated carboxy terminus. The diameter of the inhibition zones represents the antimicrobial activity; a diameter of 2.5 mm is the diameter of an empty punched well in an agar plate which contains only carrier fluid (negative control). The experiments were repeated at least three times, and the mean plus standard deviation is shown.

FIG. 2 shows the investigation of various embodiments of the peptide according to the present invention as an antibiotic against various pathogens. The following peptides were investigated: modified octapeptide (SEQ ID no. 4), wild type octapeptide (SEQ ID no. 1), alanine-mutated octapeptide (SEQ ID no. 7), and a heptapeptide (SEQ ID no. 8) (each 50 μg/ml) were tested in a flow cytometric antimicrobial effectiveness assay against Escherichia coli, Staphylococcus aureus, Candida albicans, and Bacteroides fragilis. The letters once again indicate the respective amino acids in the one-letter code. The experiments were repeated twice in double batches, and the mean plus standard deviation is shown;

FIG. 3 shows the results of investigations of an octapeptide according to the present invention that is made up of D-amino acids, compared with an octapeptide made up of L-amino acids, with respect to E. coli K12 (a) and Bifidobacterium adolescentis (b).

FIG. 4 shows the results of further investigations of the activity of various octapeptides according to the present invention with respect to pathogenic bacteria and fungi in a radial diffusion assay.

FIG. 5 shows the results of investigations of the cell toxicity of the octapeptides on the intestinal cell line CaCo-2.

### DETAILED DESCRIPTION

As stated initially, antimicrobial peptides (AMPs) are produced by almost all organisms and represent an initial barrier to microbial infection. Many AMPs exhibit antimicrobial activity against both Gram-positive and Gram-negative bacteria, and against fungi and some viruses having coats. Humans produce different classes of AMPs, one of which, as also already mentioned above, is defensins. These are notable for their small size (3 to 5 kDa), a net cationic charge, and six conserved cysteine residues that are interconnected via three disulfide bridges. Defensins are subdivided into alpha- and beta-defensins depending on the connectivity of these bridges. To date only four beta-defensins (hBD-1 to hBD-4) have been functionally investigated, including as antibiotically effective candidates.

To date, however, the chemical synthesis of beta-defensins, which, as already mentioned earlier, have three native disulfide bridges, has represented a considerable challenge in terms of both cost and the complexity of the manufacturing method.

The peptide made available for the first time with the present invention represents an octapeptide of the C-terminal end of the defensin hBD-1, which contains two free cysteines and has proven in terms of its antimicrobial activity to be superior as compared with hBD-1 and with shorter peptide sequences from the C-terminus of hBD-1, as shown by the experiments presented below.

Bacterial and Fungal Strains

The bacterial strains Bifidobacterium adolescentis Ni3, 29c (clinical isolate), Bifidobacterium breve PZ1343, Bifidobacterium longum DSM 20219T (clinical isolate), Lactobacillus acidophilus PZ1138 (clinical isolate), Lactobacillus fermentum PZ1162 (clinical isolate), and Streptococcus salivarius spp. thermophiles DSM20617 were obtained from Ardeypharm (Germany), and Bacteroides vulgates DSM1447 was provided by DSMZ (Deutsche Sammlung für Mikroorganismen and Zellkulturen [German Microorganism and Cell Culture Collection]). The Candida albicans strain 526 was isolated from feces and was furnished by the Institut der Labormedizin, Klinik am Eichert [Laboratory Medicine Institute, Eichert Clinic] (Göppingen, Germany). Reference strains of the American Type Culture Collection (ATCC) Escherichia coli ATCC25922, Staphylococcus aureus ATCC25923 and Bacteroides fragilis ATCC25285 were furnished by the Institut der Labormedizin, Klinik am Eichert (Goppingen, Germany). The strains Enterococcus faecalis ATCC29212, Candida albicans ATCFC 10231, and Pseudomonas aeruginosa ATCC27853, obtainable from the American Type Culture Collection under the ATCC numbers indicated, were also tested.

Peptides

Human beta-defensins were obtained from Peptide Institute Inc., Osaka, Japan; carboxy-terminal heptapeptides and octapeptides, as well as reduced hBD-1, were chemically synthesized (EMC Micro Collections, Tübingen, Germany).

Antimicrobial Assays

Antimicrobial radial diffusion assays for anaerobic bacteria were carried out as described previously (see Schroder et al.: “Reduction of disulfide bonds unmasks potent antimicrobial activity of human beta-defensin 1”, Nature, 469: 419-423 (2011)). In brief, the bacteria were anaerobically cultured (Oxoid AnaeroGen™, England) for 24 hours at 37° C. on Columbia agar plates, then inoculated into liquid trypticase soy broth (TSB) medium and cultured again for 24 hours. The bacterial cultures were then washed and diluted to an optical density (OD_{620 nm})=0.1, of which 150 μl was used for the effectiveness assay. Incubation occurred under anaerobic conditions in 10 ml 10 mM sodium phosphate having a pH of 7.4 with 0.3 mg/ml TSB powder and 1% (w/v) low-EEO agarose (agarose with very low EEO value) (Appli-Chem) with 0 or 2 mM dithiothreitol (DTT, Sigma Aldrich), with 1 μg synthetic, oxidized hBD-1 (Peptide Institute, Japan) or synthetic peptides, for three hours. An overcoating gel having 6% (w/v) TSB powder, 1% agarose, and 10 mM sodium phosphate buffer (pH 7.4 or 5.7), with or without DTT, was placed onto the plates. After incubation for 48 hours at 37° C. the diameters of the inhibition zones were measured. The experiments were repeated at least three times.

Flow cytometric antimicrobial assays with which the membrane depolarization of the bacteria and fungi were measured were carried out as previously described (see Nuding et al., “A flow cytometric assay to monitor antimicrobial activity of defensins and cationic tissue extracts,” Journal of Microbiological Methods, 65: 335-380 (2006)).

In brief, 1.5×10⁶ cells per ml were incubated in 1:6-diluted Schaedler medium at 37° C. with peptides at a final volume of 50 μl. The defensins were dissolved in 0.01% acetic acid and were added to the bacterial/fungal suspensions at the final concentrations indicated. Bacterial or fungal suspensions that had been incubated with solvent (0.01% acetic acid) served here as controls for viability. After 90 minutes the suspensions were incubated for 10 minutes with 1 mg/ml of the membrane-potential-sensitive dye DiBAC4(3) ([bis-(1,3-dibutylbarbiturate)trimethine oxanol]) (Invitrogen, USA). The suspensions were centrifuged, and sediments resuspended in 300 ml phosphate-buffered saline. The percentage of depolarized fluorescing bacteria or fungi in the suspension was determined using a FACSCalibur flow cytometer (Becton-Dickinson, USA) utilizing Cell Quest software (Becton-Dickinson). The experiments were repeated twice, each in duplicate.

HPLC Analysis

For analysis by high performance liquid chromatography (HPLC), the octapeptides were mixed with 0.1% (v/v) trifluoracetic acid (TFA) and analyzed with an Agilent 1200 system (Agilent) and a Synergi reversed phase (RP) column (250×4.6 mm, 4 μm, Phenomenex, Germany). The gradient had a slope from 0% B to 12% B within 24 minutes (solvent A: water+0.18% (v/v) TFA; solvent B: acetonitrile+0.15% (v/v) TFA) at 25° C. and 0.8 ml/min.

Ion Inhibition Assay

0.25 μg/ml of the peptides or defensins were incubated at room temperature for 45 minutes with 4.5 mM NaCl, magnesium chloride MgCl₂, iron chloride FeCl₂, zinc chloride ZnCl₂, or zinc sulfate ZnSO₄. The mixture was then analyzed in radial diffusion assays in terms of its antimicrobial activity against Bifidobacterium adolescentis and Escherichia coli. The experiments were repeated at least three times.

Results

Schroder et al. (“Reduction of disulfide bonds unmasks potent antimicrobial activity of human beta-defensin 1”, Nature, 469: 419-423 (2011)) have recently shown that human beta-defensin 1 exhibits elevated antimicrobial activity under reducing conditions.

In the present case hBD-1 and its antimicrobial activity have been further investigated. For this, the antimicrobial activity of the three human beta-defensins hBD-1, -2, and -3 with respect to commensal bacteria of the human intestinal flora were tested under standard conditions (pH 7.4) and slightly acidic conditions (pH 5.7); under both conditions, reducing conditions were also tested by adding 2 mM of the chemical reducing agent dithiothreitol (DTT) to the growth medium. It was found in this context (data not shown) that with most bacteria the activity of the beta-defensins was highest under standard conditions, with the exception of hBD-1, which was largely inactive under those conditions and became active by reduction. This activation was not observed, however, at a pH of 5.7. In contrast to this, hBD-2 proved unable to be influenced by reduction, whereas a change in pH had a very negative effect on its antimicrobial activity.

In most cases hBD-3 had the strongest activity against the tested commensals, as compared with the other two defensins.

In summary, it can be stated that factors of the surrounding medium, for example redox potential and pH, can modulate the antimicrobial activity of beta-defensins against commensal intestinal bacteria. This modulation appears to be specific to individual defensin-bacteria relationships, however, and does not correlate either with Gram status or with the bacterial genus.

Experiments with a heptapeptide that represents the seven terminal amino acids of hBD-1, which already exhibits antimicrobial activity against Bifidobacterium adolescentis, have shown that the carboxy-terminal heptapeptide of the wild type had the highest activity against Bifidobacterium adolescentis, whereas peptides having an opposite amino-acid sequence were less active. Replacing the cysteine residues with alanine caused activity to be completely suppressed. The isolated amino terminus of hBD-1 was inactive.

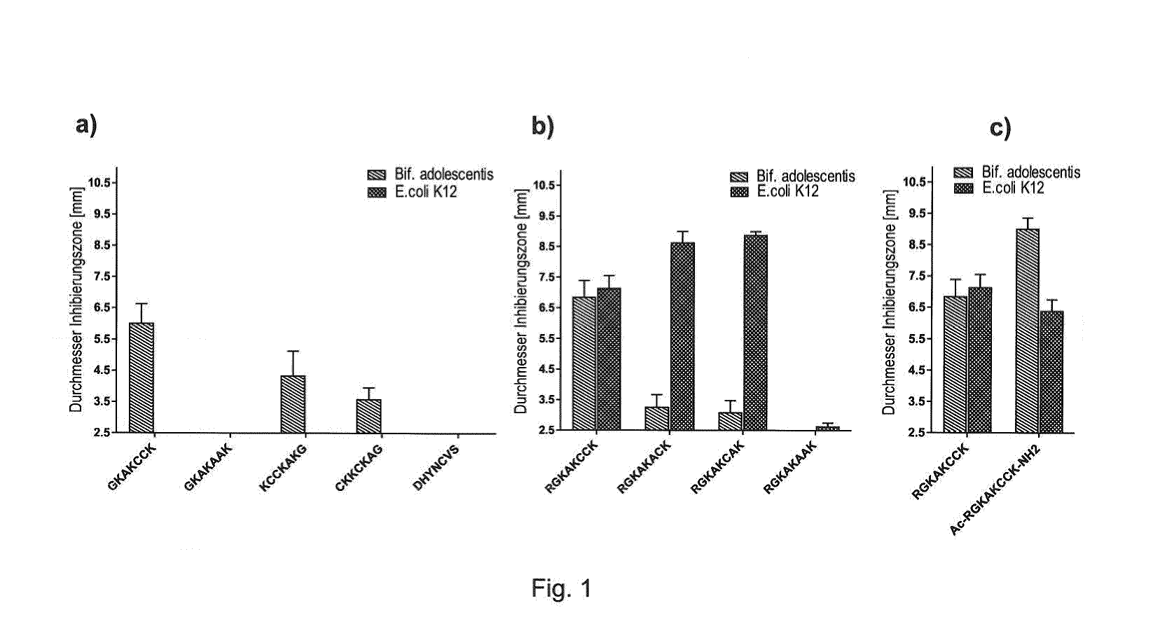

In the present case the peptide according to the present invention, an octapeptide that encompasses the eight terminal amino acids of the carboxy terminus of hBD-1, was tested next; it exhibited greater antimicrobial activity than the previously tested heptapeptide (see FIG. 1). When either Cys₆ or Cys₇ was exchanged, activity was greatly decreased with respect to Bifidobacterium adolescentis to the same extent, whereas exchanging both cysteines brought activity to a complete standstill. In contrast to the previously tested heptapeptide, the octapeptide also had antimicrobial activity against Escherichia coli (see FIG. 1b). Surprisingly, exchanging Cys₆ or Cys₇ for alanine in this case increased the antimicrobial activity, whereas exchanging both cysteines again almost entirely shut down antimicrobial activity.

In order to optimize the octapeptide and improve its stability with respect to proteases, in a subsequent step the amino terminus of the octapeptide was stabilized by acetylation, and the carboxy terminus by amidation. While activity with respect to Escherichia coli did not differ significantly by comparison with the wild type peptide, the activity against Bifidobacterium adolescentis rose sharply (see FIG. 1c).

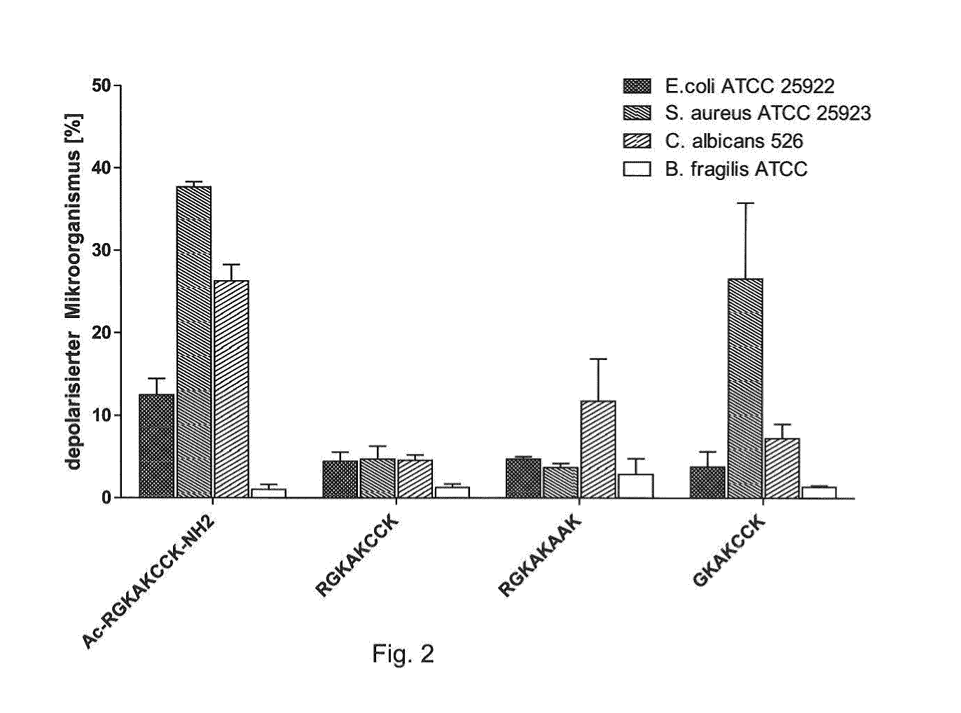

Using the newly identified and furnished octapeptide, an easily and economically manufacturable peptide having antibiotic effects, which can be used as a therapeutic agent, is made available. Both the modified and unmodified peptides were therefore investigated in terms of their ability to kill (opportunistic) pathogenic microorganisms. Flow cytometry assays were performed for this purpose (see FIG. 2), and these showed that the effectiveness of the wild type octapeptide and alanine-mutated peptide and of the wild type heptapeptide was only marginal in most cases. In contrast thereto, the modified octapeptide had outstanding activity against the pathogenic microorganisms Staphylococcus aureus and Candida albicans, but not with respect to Escherichia coli and Bacteroides fragilis.

It has thus become apparent that stabilization of the termini increases antimicrobial activity not only with respect to the commensal intestinal bacterium Bifidobacterium adolescentis but also with respect to at least two pathogenic microorganisms of clinical relevance.

In further experiments a reversed-phase HPLC analysis was carried out in order to investigate the hydrophobicity of the tested peptides (data not shown). The modified peptide was the last to elute from the column, indicating the highest hydrophobicity; it was preceded by elution of the wild type peptide, the individual alanine-amino acid exchange variants, and the double alanine-amino acid exchange variants (data not shown).

In order to further investigate the role of charge and of ion interactions, oxidized and reduced hBD-1 and the wild type and modified octapeptide were incubated with monovalent and divalent cations (data not shown). In terms of Escherichia coli, the activity of the complete defensin was completely shut down by pre-incubation with magnesium chloride or iron chloride, whereas the activity of the carboxy-terminal peptides was greatly inhibited but still detectable after pre-incubation with these metal ions. In contrast thereto, NaCl did not influence antimicrobial activity with respect to E. coli.

The investigation of Bifidobacterium adolescentis showed again that pre-incubation with sodium chloride did not have a strong effect on activity, whereas iron chloride, zinc chloride, and zinc sulfate completely abolished or greatly reduced activity. Unlike with E. coli, incubation with magnesium chloride did not influence antibiotic activity against Bifidobacterium.

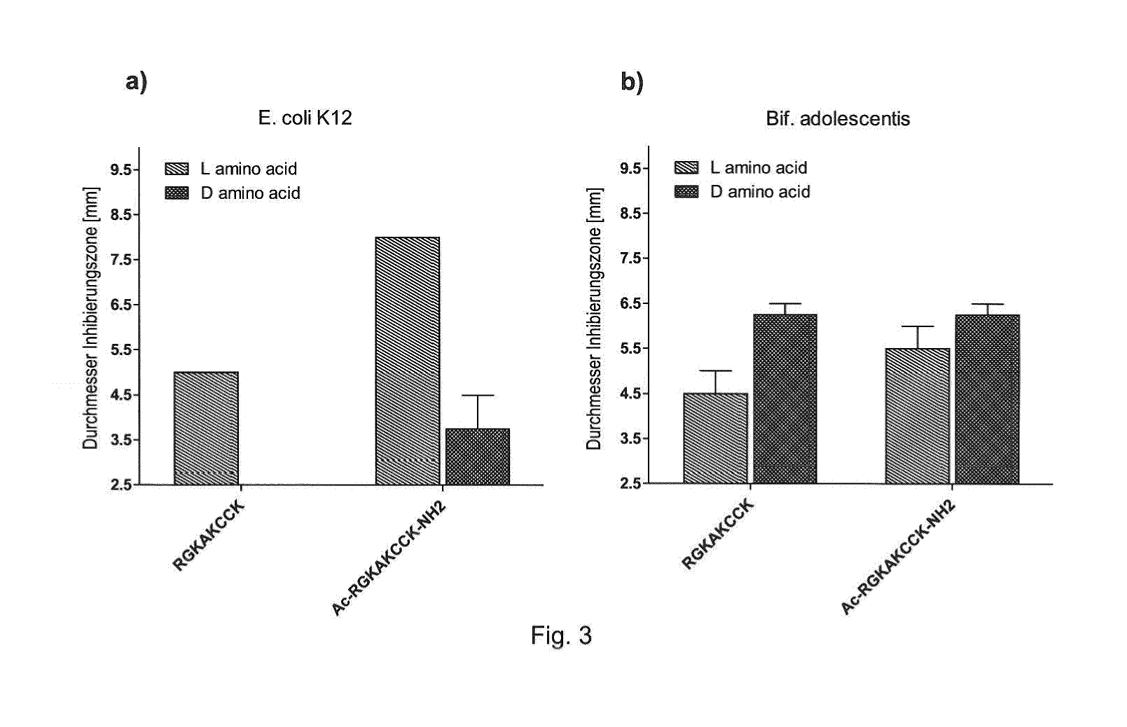

In further experiments, an octapeptide according to the present invention having the sequence RGKAKCCK (SEQ ID no. 1) made up of D-amino acids (except glycine) was investigated by comparison with an octapeptide having the sequence RGKAKCCK (SEQ ID no. 1) made up of L-amino acids, the termini being modified and unmodified (FIG. 3). With respect to E. coli K12 (see FIG. 3a), the peptide made up of D-amino acids and having modified termini (N-terminus: acetylated; C-terminus: amidated) exhibited weaker activity, whereas this peptide in both unmodified form and modified form had elevated activity, with respect to Bifidobacterium adolescentis as compared with the peptide made up of L-amino acids (see FIG. 3b).

These data therefore show in total that the interaction between peptide and cations is not based only on a positive charge, but instead that specific ions can influence activity against specific bacteria.

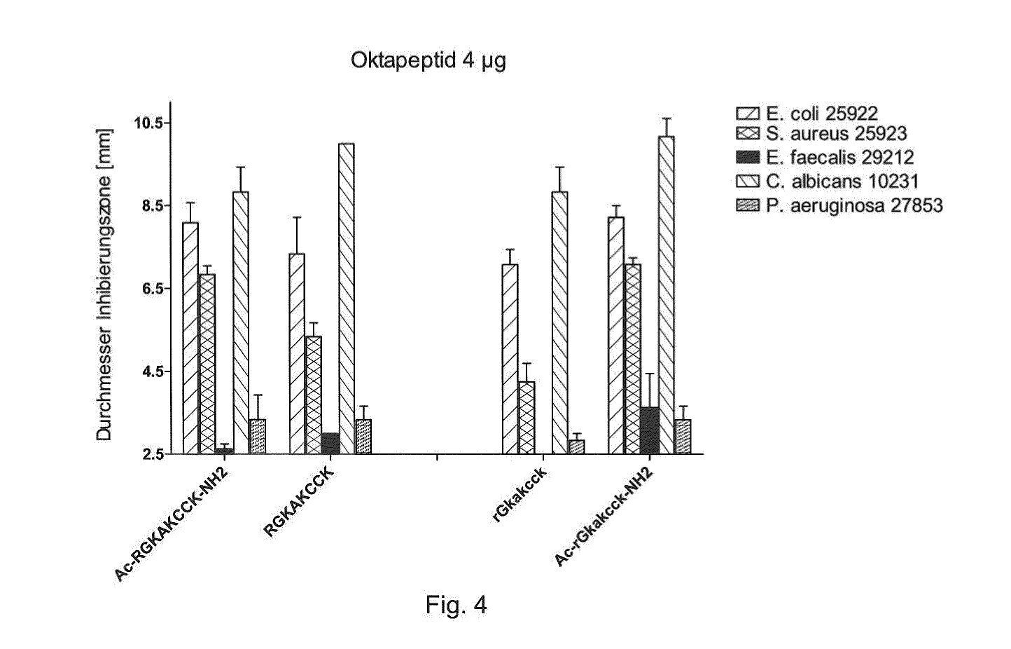

The activity of the octapeptides according to the present invention against pathogenic bacteria and fungi was also confirmed in further radial diffusion assays: activity was tested against the strains Escherichia coli 25922, Staphylococcus aureus 25923, Enterococcus faecalis 29212, Candida albicans 10231, and Pseudomonas aeruginosa 27853 (see FIG. 4), using the octapeptide according to the present invention having the sequence RGKAKCCK (SEQ ID no. 1) made up either of L-amino acids (see FIG. 4; five bars on the left of the diagram) or of D-amino acids (except glycine) (see FIG. 4; five bars on the right of the diagram), on the one hand with modified and on the other hand with unmodified termini (N-terminus: acetylated; C-terminus: amidated). In addition to outstanding activity against Escherichia coli and Staphylococcus aureus, this also revealed in particular excellent activity with respect to Candida albicans on the part of all variants of the tested octapeptide.

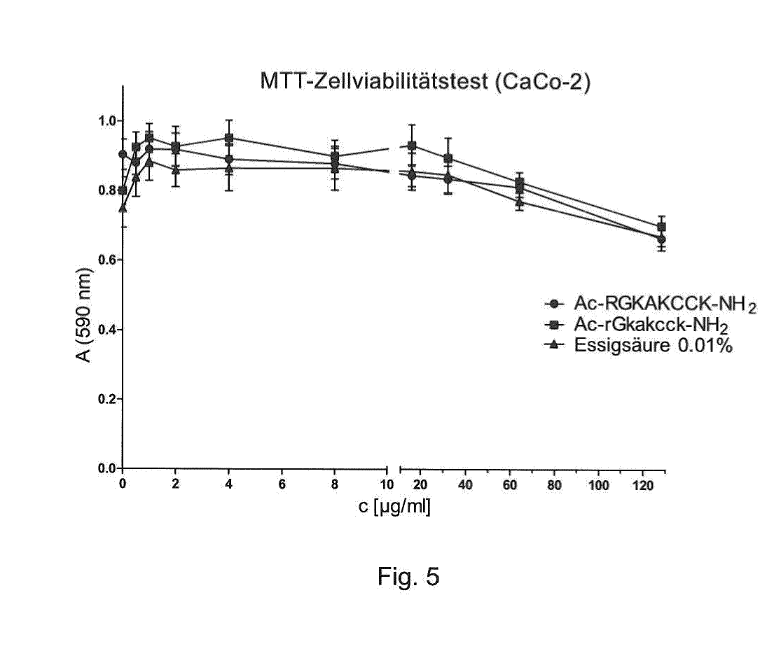

The cell toxicity of the terminally stabilized octapeptides was also investigated in further experiments with MTT-((3-[4,5-dimethylthiazol-2-yl]-2,5-diphenyltetrazolium bromide; thiazolyl blue), specifically with respect to the human cell line CaCo2 (ATCC HTB-37), using increasing concentrations of the respective octapeptides (SEQ ID no. 1; L- or D-amino acids (except glycine) having modified termini (N-terminus: acetylated; and C-terminus: amidated)), compared with increasing concentrations of 0.01% acetic acid. The results thereof are reproduced in FIG. 5, showing that the stabilized octapeptides possessed no cell toxicity exceeding that of the 0.01% acetic acid solvent. The octapeptides according to the present invention are thus also suitable for use in therapeutic applications.

The results and data presented above clearly show, however, that the octapeptide made available here for the first time, in the wild type form, with an amino acid exchange, and/or in stabilized form, is an outstanding agent having antibiotic effectiveness. These results are surprising, and were not to be expected based on the existing art hitherto available.

1-12. (canceled)

13. A peptide or derivative thereof that has antimicrobial activity and has a C-terminus and an N-terminus, and that is made up of at least eight and at most 12 successive amino acids, the peptide exhibiting the sequence having the following formula I:
Ter₁-X₁-B₁-X₂-B₂-X₃-Z₁-Z₂-X₄-Ter₂ (formula I)
in which
Ter₁ is the free N-terminal amino group of the N-terminal amino acid X₁, or a modified N-terminal amino group;
X₁, X₂, and X₃ are each identical or different and are selected, mutually independently in each case, from an amino acid having a basic side chain;
B₁ and B₂ are identical or different and are each an amino acid having an aliphatic or basic side chain;
Z₁ and Z₂ either are each cysteine, or are cysteine and alanine; and
Ter₂ is the free C-terminal carboxyl group of the C-terminal amino acid X₄, or is a modified C-terminal carboxyl group.

14. The peptide of claim 13, wherein it is made up of eight sequential amino acids, and it possesses a sequence of formula I.

15. The peptide of claim 13, wherein the peptide is selected from one of SEQ ID nos. 1 to 6 or derivatives thereof, the derivatives being formed by exchanging at least one amino acid with a derivative of the amino acid.

16. The peptide of claim 13, wherein it is made up of D-amino acids or L-amino acids or of mixtures thereof.

17. The peptide of claim 13, wherein the peptide is modified at the C-terminus and/or N-terminus by an acetylation, amidation, formylation, phosphorylation.

18. The peptide of claim 13, wherein it is used as an antibiotic, and/or in a disinfecting agent or cleaning agent.

19. The peptide of claim 18, wherein for the treatment and/or prophylaxis of inflammatory or infectious diseases that are caused by microorganisms.

20. The peptide of claim 19, wherein, wherein the inflammatory or infectious disease is caused by a microorganism that is a bacterium, a virus, or a yeast.

21. The peptide of claim 18, wherein the inflammatory or infectious disease is caused by a microorganism that is selected from Bifidobacterium sp., Lactobacillus sp., Escherichia coli, Streptococcus sp., Staphylococcus sp., Bacteroides sp., Candida sp., Pseudomonas sp., Propionibacterium sp., Treponema sp.

22. The peptide of claim 18, wherein, wherein the inflammatory or infectious disease is selected from chronic inflammatory intestinal diseases, inflammatory diseases of the oropharyngeal cavity, pulmonary diseases, diseases of the urogenital tract, diseases of the pancreas, diseases of the female reproductive system, diseases of or injuries to or burns of the skin.

23. A pharmaceutical composition, comprising:
a pharmaceutically acceptable carrier; and
at least one peptide or derivative thereof that has antimicrobial activity and has a C-terminus and an N-terminus, and that is made up of at least eight and at most 12 successive amino acids, the peptide exhibiting the sequence having the following formula I:
Ter₁-X₁-B₁-X₂-B₂-X₃-Z₁-Z₂-X₄-Ter₂ (formula I)
in which
Ter₁ is the free N-terminal amino group of the N-terminal amino acid X₁, or a modified N-terminal amino group;
X₁, X₂, and X₃ are each identical or different and are selected, mutually independently in each case, from an amino acid having a basic side chain;
B₁ and B₂ are identical or different and are each an amino acid having an aliphatic or basic side chain;
Z₁ and Z₂ either are each cysteine, or are cysteine and alanine; and
Ter₂ is the free C-terminal carboxyl group of the C-terminal amino acid X₄, or is a modified C-terminal carboxyl group.

24. A polynucleotide coding for at least one peptide or derivative thereof that has antimicrobial activity and has a C-terminus and an N-terminus, and that is made up of at least eight and at most 12 successive amino acids, the peptide exhibiting the sequence having the following formula I:
Ter₁-X₁-B₁-X₂-B₂-X₃-Z₁-Z₂-X₄-Ter₂ (formula I)
in which
Ter₁ is the free N-terminal amino group of the N-terminal amino acid X₁, or a modified N-terminal amino group;
X₁, X₂, and X₃ are each identical or different and are selected, mutually independently in each case, from an amino acid having a basic side chain;
B₁ and B₂ are identical or different and are each an amino acid having an aliphatic or basic side chain;
Z₁ and Z₂ either are each cysteine, or are cysteine and alanine; and
Ter₂ is the free C-terminal carboxyl group of the C-terminal amino acid X₄, or is a modified C-terminal carboxyl group.

25. The peptide of claim 13, wherein X₁, X₂, and X₃ are each identical or different and are selected, mutually independently in each case, from an amino acid having a basic side chain, are selected from one of the following: arginine, lysine, 6-hydroxylysine, homoarginine, 2,4-diaminobutyric acid, [beta]-homoarginine, D-arginine, arginal, 2-amino-3-guanidinopropionic acid, nitroarginine, n-methylarginine, [epsilon]-n-methyllysine, allo-hydroxylysine, 2,3-diaminopropionic acid, 2,2′-diaminopimelic acid, ornithine, sym-dimethylarginine, asym-dimethylarginine.

26. The peptide of claim 13, wherein B₁ and B₂ are identical or different and are each an amino acid having an aliphatic or basic side chain, and are selected from alanine or glycine.

27. The peptide of claim 13, wherein X₁, X₂, and X₃ are each identical or different and are selected, mutually independently in each case, from an amino acid having a basic side chain, are selected from one of the following: arginine, lysine, 6-hydroxylysine, homoarginine, 2,4-diaminobutyric acid, [beta]-homoarginine, D-arginine, arginal, 2-amino-3-guanidinopropionic acid, nitroarginine, n-methylarginine, [epsilon]-n-methyllysine, allo-hydroxylysine, 2,3-diaminopropionic acid, 2,2′-diaminopimelic acid, ornithine, sym-dimethylarginine, asym-dimethylarginine, and wherein B₁ and B₂ are identical or different and are each an amino acid having an aliphatic or basic side chain, and are selected from alanine or glycine.


SEQUENCE LISTING (12 sequences)

SEQ ID NO: 1 - protein, 8 aa, Artificial Sequence
  Synthetically manufactured peptide, including sequences of the C-Terminus of hBD-1
RGKAKCCK

SEQ ID NO: 2 - protein, 8 aa, Artificial Sequence
  Synthetic peptide with antimicrobial activity
RGKAKCAK

SEQ ID NO: 3 - protein, 8 aa, Artificial Sequence
  Synthetic peptide with antimicrobial activity
RGKAKACK

SEQ ID NO: 4 - protein, 8 aa, Artificial Sequence
  Synthetic Peptide with modified Termini
RGKAKCCK

SEQ ID NO: 5 - protein, 8 aa, Artificial Sequence
  Synthetic Peptide with modified Termini
RGKAKCAK

SEQ ID NO: 6 - protein, 8 aa, Artificial Sequence
  Synthetic Peptide with modified Termini
RGKAKACK

SEQ ID NO: 7 - protein, 8 aa, Artificial Sequence
  Synthetic Peptide
RGKAKAAK

SEQ ID NO: 8 - protein, 7 aa, Artificial Sequence
  Synthetic Peptide
GKAKCCK

SEQ ID NO: 9 - protein, 7 aa, Artificial Sequence
  Synthetic Peptide
GKAKAAK

SEQ ID NO: 10 - protein, 7 aa, Artificial Sequence
  Synthetic Peptide
KCCKAKG

SEQ ID NO: 11 - protein, 7 aa, Artificial Sequence
  Synthetic Peptide
CKKCKAG

SEQ ID NO: 12 - protein, 7 aa, Artificial Sequence
  Synthetic Peptide
DHYNCVS